(19) United States
(12) Patent Application Publication
(10) Pub. No.: US 2009/0131648 A1
(43) Pub. Date: May 21, 2009

(54) Rigid dendrimeric structures

(76) Inventor: Mikhail S. Shchepinov, Oxford (GB)

Correspondence Address: WENDEROTH, LIND & PONACK, L.L.P., 1030 15th Street, N.W.,, Suite 400 East, Washington, DC, 20005-1503, US

(21) Appl. No.: 12/149,319

(22) Filed: Apr. 30, 2008

Related U.S. Application Data
(60) Provisional application No. 60/924,095, filed on Apr. 30, 2007.

(51) Int. Cl. C07H 21/00; C08G 65/00; C08G 73/00

(52) U.S. Cl. 536/25.3; 528/425; 528/422; 528/392; 536/22.1

(57) ABSTRACT

The present invention provides novel dendrimers having a multimeric structure obtainable by reaction of a core molecule having an adamantoid structure with branching synthons and terminating synthons, said dendrimers immobilised on a support and a method of hybridisation which uses said dendrimers immobilised on a support.

All documents cited herein are incorporated by reference in their entirety.

### TECHNICAL FIELD

The present invention relates to the production of branched dendrimeric structures having a core structure which constrains orientation of the branched chains.

### BACKGROUND OF THE INVENTION

Since their first synthesis, dendrimers have attracted considerable attention as a new branch of polymer science. Two basic strategies for the synthesis of these structures have been proposed, specifically: divergent, with the structure grown up from the centre to the periphery; and convergent, wherein the structure is grown from the periphery to the centre. WO-A-99/10362 describes dendrimers having a multimeric structure which are obtained by reacting a core molecule with branching synthons and optionally non-branching synthons.

One of the most important parameters governing a dendrimeric structure and its generation, is the number of branches generated at each step. This defines the number of repetitive steps necessary to build up the desired molecule and the density of the groups at the periphery. The main properties of a dendrimeric structure are determined by the functional end groups of moieties on its outer shell. Many applications proposed for dendrimers exploit the high density and the large number of functional end groups. For example, dendrimers with a positively charged outer surface interact strongly with nucleic acid, a property which has been used recently for the transport of nucleic acids through the membranes of living cells; see Boussif et al, Proc. Natl. Acad. Sci., USA, (1995):92:7297-7301.

Branched (dendrimeric) polynucleotides can be used to amplify radioactive or fluorescent signals in hybridisation tests. Such amplification may be particularly important in in situ hybridisation and in the emerging techniques which exploit oligonucleotide arrays, where the signal is limited by the surface density of the oligonucleotides or the target molecule.

However, there are several disadvantages associated with known dendrimers. The branches of known dendrimers are generally flexible. This means that the functional end-groups occasionally come into a contact. This particularly causes problems where tags such as fluorophores are attached to the end-groups, as it may lead to quenching as is described in Streibel et al, Experim and Mol. Path, 2004, 77, 89. In addition, even in the case where the functional end groups do not come into contact, the close proximity between the groups may cause problems. For example, where the dendrimer is a dendrimeric polynucleotide, the quality of detection and the hybridisation yield may be compromised as a consequence of there not being sufficient space for the target DNA molecule to contact the polynucleotide and hybridise.

There is hence a need for new dendrimer structures, in particular for use in controlling the hybridisation of polynucleotides onto microarrays which overcome the aforementioned disadvantages. Specifically, there is a need for dendrimer structures wherein the spacing of the branches is controlled to optimise detection and hybridisation yield when used in hybridisation tests.

### SUMMARY OF THE INVENTION

In this regard, the present invention provides novel dendrimers produced by reaction of a rigid core molecule with branching and terminating synthons. Advantageously, the present inventors have found that by careful selection of the core structure, it is possible to control the spatial density of the resulting dendrimer. This means that the dendrimers obtained are particularly useful when employed to amplify radioactive or fluorescent signals in hybridisation experiments. More specifically, by selecting a core molecule which has a rigid structure, contact between functional end groups of the dendrimer is avoided and, in addition, the spacing between adjacent end groups is optimised to allow passage of, for example, a DNA molecule between adjacent groups.

### BRIEF DESCRIPTION OF THE FIGURE

FIG. 1 illustrates advantages associated with using dendrimers according to the present invention in hybridisation techniques as compared to known dendrimers.

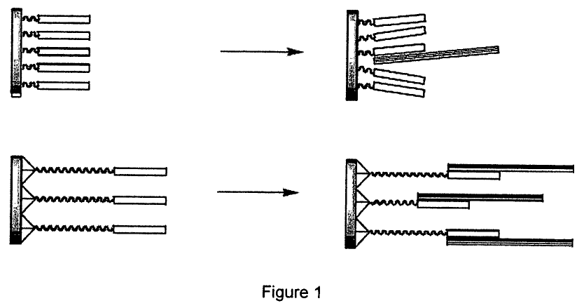

### DETAILED DESCRIPTION OF THE INVENTION

More specifically, the present invention provides a dendrimer having a multimeric structure, obtainable by the reaction of a core molecule with branching synthons and terminating synthons, wherein the dendrimer has the formula (1):

X—[(Y)ₙ-(Z)ₘ]ₚ (1)

wherein X is a core molecule having an adamantoid structure; Y is a branching synthon; Z is a terminating synthon; n and m are independently integers having a value in the range from 1 to 50; and p is an integer in the range from 1 to 10.

In a second aspect, the present invention relates to a dendrimer as defined above immobilised on a solid support.

In a third aspect, the present invention provides a method of hybridisation wherein a nucleic acid sample is contacted with a dendrimer according to the present invention, having outer ends which terminate with a polynucleotide sequence immobilised on a solid support, under hybridising conditions, wherein binding of the nucleic acid sample of interest to the polynucleotide ends of the dendrimer triggers a detectable signal.

Preferably the dendrimers of the present invention are synthesised using the divergent synthesis technique wherein branches are built up sequentially. This is a technique with which the skilled person is familiar.

A feature of a dendrimer according to the present invention is that it has multiple termini and only a single path can be traced through its branches from one terminus to another. The skilled person will appreciate that in tracing the path from one terminus to another, there are a number of different paths which can be taken through the core molecule which has an adamantoid structure.

Advantageously, in the dendrimers of the present invention, at least one unit in each of each of Y and Z may contain a functional group which is labelled. Examples of suitable labelling groups include fluorophores such as dyes, quenchers which have they effect of quenching fluorescence and mass tags. As used herein, the term “mass tag” refers to compounds which enhance mass spectrometry by improving the ionisation properties of the molecules being analysed. Examples of particularly useful mass tags are trityl derivatives including:

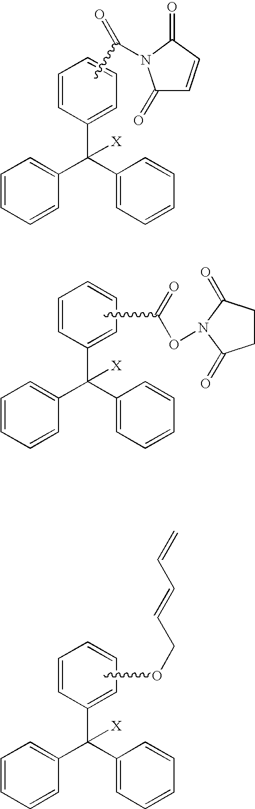

Advantageously, a dendrimer according to the present invention may comprise a number of different groups Y and Z meaning that a single dendrimer may be used for a number of different purposes. More specifically, a single dendrimer can contain a number of different functionalities making it versatile and appropriate for use in a number of different applications.

The branches [(Y)ₙ-(Z)ₘ] of the dendrimers of the present invention need not be of the same length. In this regard, by including branches having a variety of lengths, it is possible to produce a dendrimer having a surface that is concave or convex.

Advantageously, the dendrimers of the present invention have branch span of greater than about 15 Å. The branch span of a dendrimer is defined herein as the distance between end groups on adjacent branches of the dendrimer where the branches in question are fully stretched, as determined by X-ray crystallography where a crystal structure is available or molecular modelling in the absence of such a structure.

### X

X is the core molecule to which all of the p branches which form the dendrimer structure will be attached. Advantageously, in order to provide a dendrimer which has an optimised surface density and arrangement, X has an adamantoid structure.

As used herein, the term “adamantoid” is used to describe a saturated, polycyclic cage structure which is based on that of adamantane. Adamantane (tricyclo[3.3.1.1^{3,7}]decane) is formed from four fused cyclohexane rings and has the rigid structure shown below:

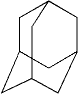

Examples of other compounds which have an adamantoid structure and are therefore suitable for use as the core molecule, X, in the present invention include P₄O₆, As₄O₆, P₄O₁₀ (═(PO)₄O₆), P₄S₁₀(═(PS)₄S₆), and N₄(CH₂)₆.

Advantageously, an adamantoid structure has a number of sites at which chemical growth can be initiated. By use of a core molecule having an adamantoid structure, the growing chains are directed away from each other and contact between them is minimised.

As the skilled person will appreciate, the maximum number of synthons which can be grown on a core molecule having the above adamantoid structure is 10.

The core molecule should be chemically inert to the synthetic procedures used during the synthesis of the dendrimer and to any conditions to which it will be exposed during its end use.

### Branching Synthons, Y

The dendrimer of the present invention is formed by reacting the core molecule with branching synthons and terminating synthons. Methods for synthesizing dendrimers are well-established as described, for example in WO-A-99/10362. The skilled person is therefore readily familiar with such techniques.

The term “branching synthon” as used herein refers to a building unit which comprises at least three coupling sites which are joined at a junction.

Preferably the branching synthon Y of the dendrimer of the present invention is obtained by reacting a synthon of formula (2) with the core molecule (X) or a growing branch of the dendrimer:

(P¹)F¹-L¹-J[-L²-F²(P²)]_{q} (2)

wherein F¹P¹ and F²P² are each independently an optionally protected functional group; L¹ and L² are linker groups, J is a junction group; and q is an integer in the range from 2 to 10.

Thus the branching synthon Y in formula (1) is a residue of a synthon of formula (2).

### F¹ and F²

The nature of the functional groups F¹ and F² will determine the chemical properties of the synthons. The functional groups F¹ and F² may be the same or different and are preferably selected such that F² is reactive with F¹ such that where a growing branch of the dendrimer terminates with an F² group, the F¹ group on a synthon of formula (2) which is reacted with the growing branch, reacts with the F² group to attach to the growing branch.

The specific nature of the functional groups F¹ and F² will depend on the end application of the dendrimer. Examples of suitable functional groups include hydroxyl, amino, sulphydryl; phosphate, phosphitamide groups or derivatives thereof.

### P¹ and P²

During synthesis, the groups F¹ and F² are optionally protected to prevent unwanted side reactions. There are a wide variety of suitable protecting groups with which the skilled person will be familiar. Preferred protecting groups P¹ and P² include, for example, trityl alcohols (such as DMTr), fluoroenylmethoxy carbonyl groups or levulinyl groups.

Advantageously, P¹ and P² may be selected to be removed by different conditions such that it is possible to remove some protecting groups while leaving others in place. In this way, it is possible to control the sequence in which particular branches are grown on the core molecule. In particular, during synthesis, it is group F² which reacts with the subsequent synthons. Therefore, it is advantageous that F² remains protected during coupling.

The protecting groups used in the present invention may be those which are removed by one or more of light, acid, base, fluoride ions, hydrazine and hydrogenation.

### L¹ and L²

L¹ and L² are linker groups which join the functional groups to the junction J. These linker groups are selected having regard to a number of different properties, including synthon length, solvation properties, degree of branching, flexibility, chirality and chemical functionality. The different properties associated with different chemical moieties are well understood by the skilled person and thus when faced with a particular application, the skilled person would readily be able to select appropriate linker groups. Examples of suitable linker groups include, but are not limited to, polymeric chains of hydrocarbyl, hydrocarbylene, heterohydrocarbyl, heterohydrocarbylene, aromatic and hydrocarbyloxy residues.

Preferably at least one of L¹ and L² is an alkoxy group.

Preferably, in order to obtain a rigid dendrimer structure, the spatial rotation of the branching synthons used in the present invention is preferably restricted by selection of a synthon which contains at least one unsaturated C-M bond, wherein M is selected from C or N. In this regard, preferably the branching synthon used to obtain the dendrimer of the present invention contains at least one of a C₂₋₄alkenylene, C₂₋₄heteroalkenylene, C₂₋₄alkynylene, C₂₋₄heteroalkynylene, C₄₋₁₄arylene or C₅₋₁₄heteroarylene group. Advantageously, in order to maximise rigidity, preferably at least one of L¹ and L² contains a C₂₋₄alkynylene group and/or a phenylene group, preferably a C₄alkynylene, e.g. a butylene group.

Alternatively, where the branches are required to be directed at an angle, preferably at least one of L¹ and L² contains a C₂₋₄alkenylene group.

L¹ and L² may be the same or different.

L¹ and L² may be cleavable.

It may be advantageous to include a functional group in the linker in order to ensure that it has the required chemical and physical properties. Examples of such functional groups include cross-linkable groups such as sulfhydryl (S—H) and selenohydryl (Se—H) which can be cross-linked by e.g. oxidation, to form a more rigid dendrimer, charged groups and hydrogen donors and acceptors.

### J

J is a junction group by means of which the different branches of the branched synthon are joined. J is preferably a multivalent atom such as carbon, silicon, phosphorus or nitrogen or an aromatic six membered ring, such as, for example benzene.

Preferably J is a carbon atom.

An example of a preferred group of branching synthons for use in synthesising the dendrimers of the present invention are those in which, at least one of L¹ and L² contains a C₂₋₄alkynylene group and/or a phenylene group, J is a carbon atom and at least one of F¹ and F² is an optionally protected hydroxyl group.

q

q is an integer having a value in the range from 2 to 10.

Preferably q is an integer having a value in the range from 2 to 6, more preferably from 2 to 4.

n and m

n and m are independently integers having a value in the range from 1 to 50. Preferably, m is greater than n.

p

p is an integer having a value in the range from 1 to 10

Preferably p is in the range from 2 to 4, more preferably p is 4.

### Z

Z is a terminating synthon. A terminating synthon is one which is used in the last step in the synthesis of the dendrimers of the present invention. The terminating synthons used to obtain the group Z in the dendrimers of the present invention, by reacting with (X(Y)ₙ) or the growing branches of the dendrimer during synthesis, preferably have formula (2) as defined above with the exception that they may be non-branched and thus q is independently an integer having a value in the range from 1 to 10.

The end functional groups (P²F²) of such synthons will preferably form the outermost layer of the dendrimer and will therefore define its chemical and physical properties. Preferably the terminating synthons used to obtain the dendrimers of the present invention are selected to have end functional groups which are selected from charged, hydrophilic, hydrophobic, hydrogen-donor, hydrogen-acceptor, metal coordinating, reducing and oxidising groups.

In a preferred embodiment, the terminating synthons have an end functional group which is a polynucleotide, preferably selected from poly-A, poly-T, poly-G and poly-C.

The m terminating synthons may be the same or different. Preferably the m terminating synthons have a plurality of different end functional groups which are optionally blocked.

### Immobilisation

Advantageously, the dendrimers of the present invention may be immobilised on a solid support. Such immobilised dendrimers are particularly useful in methods of hybridisation.

Suitable solid supports include but are not limited to, glasses, ceramics, metals e.g. gold, polydimethylsiloxanes (PDMS) and plastics.

The dendrimers of the present invention are immobilised on a solid support by conventional techniques which are well documented. In this regard, reference is made to the techniques described in WO-A-99/10362 and in Shchepinov et al. (Nucl. Acids Res 1997 25 1155). For example, a dendrimer may be attached to a gold surface by utilising sulfhydryl groups on the dendrimer which bind covalently to the gold surface. This is a technique which is well known in the art. Alternatively, for example, immobilisation may be achieved as described in WO-A-99/10362 where the hydrophobicity of the dendrimer is modified so that it will interact with the surface and bind.

Preferably the dendrimers of the present invention immobilised on a support have end functional groups that react or terminate with a polynucleotide sequence.

### Hybridisation

As described above, dendrimers of the present invention which have end functional groups that terminate with a polynucleotide sequence are particularly useful in hybridisation experiments. The controlled spacing between the branches of the dendrimer means that hybridisation yields are improved. Additionally, as contact between branches of the dendrimer is minimised, detection is optimised.

The present invention thus provides a method of hybridisation wherein a nucleic acid sample is contacted, under hybridising conditions, with a dendrimer according to the present invention, having outer ends which terminate with a polynucleotide sequence and which is immobilised on a solid support to bind the nucleic acid sample of interest to the polynucleotide ends of the dendrimer.

The hybridising conditions under which hybridisation occurs may be conventional conditions with which the skilled person will be familiar.

Binding of the nucleic acid to the dendrimer may be monitored by one or more detectable signals such as, for example, fluorescence, luminescence, mass tags or radioactivity.

### Polynucleotides

The polynucleotides used in the present invention may be of any suitable length. In particular, the polynucleotides may be 10 to 200 nucleotides in length.

### Chemical Groups

The term ‘halogen’ includes fluorine, chlorine, bromine and iodine.

The term ‘hydrocarbyl’ includes linear, branched or cyclic monovalent groups consisting of carbon and hydrogen. Hydrocarbyl groups thus include alkyl, alkenyl and alkynyl groups, cycloalkyl (including polycycloalkyl), cycloalkenyl and aryl groups and combinations thereof, e.g. alkylcycloalkyl, alkylpolycycloalkyl, alkylaryl, alkenylaryl, cycloalkylaryl, cycloalkenylaryl, cycloalkylalkyl, polycycloalkylalkyl, arylalkyl, arylalkenyl, arylcycloalkyl and arylcycloalkenyl groups. Preferred hydrocarbyl are C₁₋₁₄ hydrocarbyl, more preferably C₁₋₈ hydrocarbyl.

Unless indicated explicitly otherwise, where combinations of groups are referred to herein as one moiety, e.g. arylalkyl, the last mentioned group contains the atom by which the moiety is attached to the rest of the molecule.

The term ‘hydrocarbylene’ includes linear, branched or cyclic divalent groups consisting of carbon and hydrogen formally made by the removal of two hydrogen atoms from the same or different (preferably different) skeletal atoms of the group. Hydrocarbylene groups thus include alkylene, alkenylene and alkynylene groups, cycloalkylene (including polycycloalkylene), cycloalkenylene and arylene groups and combinations thereof, e.g. alkylenecycloalkylene, alkylenepolycycloalkylene, alkylenearylene, alkenylenearylene, cycloalkylenealkylene, polycycloalkylenealkylene, arylenealkylene and arylenealkenylene groups. Preferred hydrocarbylene are C₁₋₁₄ hydrocarbylene, more preferably C₁₋₈ hydrocarbylene.

The term ‘hydrocarbyloxy’ means hydrocarbyl-O—.

The terms ‘alkyl’, ‘alkylene’, ‘alkenyl’, ‘alkenylene’, ‘alkynyl’, or ‘alkynylene’ are used herein to refer to both straight, cyclic and branched chain forms. Cyclic groups include C₃₋₈ groups, preferably C₅₋₈ groups.

The term ‘alkyl’ includes monovalent saturated hydrocarbyl groups. Preferred alkyl are C₁₋₁₀, more preferably C₁₋₄ alkyl such as methyl, ethyl, n-propyl, i-propyl or t-butyl groups.

Preferred cycloalkyl are C₅₋₈ cycloalkyl.

The term ‘alkoxy’ means alkyl-O—.

The term ‘alkenyl’ includes monovalent hydrocarbyl groups having at least one carbon-carbon double bond and preferably no carbon-carbon triple bonds. Preferred alkenyl are C₂₋₄ alkenyl.

The term ‘alkynyl’ includes monovalent hydrocarbyl groups having at least one carbon-carbon triple bond and preferably no carbon-carbon double bonds. Preferred alkynyl are C₂₋₄ alkynyl.

The term ‘aryl’ includes monovalent aromatic groups, such as phenyl or naphthyl. In general, the aryl groups may be monocyclic or polycyclic fused ring aromatic groups. Preferred aryl are C₆-C₁₄aryl.

Other examples of aryl groups are monovalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene.

The term ‘alkylene’ includes divalent saturated hydrocarbylene groups. Preferred alkylene are C₁₋₄ alkylene such as methylene, ethylene, n-propylene, i-propylene or t-butylene groups.

Preferred cycloalkylene are C₅₋₈ cycloalkylene.

The term ‘alkenylene’ includes divalent hydrocarbylene groups having at least one carbon-carbon double bond and preferably no carbon-carbon triple bonds. Preferred alkenylene are C₂₋₄ alkenylene.

The term ‘alkynylene’ includes divalent hydrocarbylene groups having at least one carbon-carbon triple bond and preferably no carbon-carbon double bonds. Preferred alkynylene are C₂₋₄ alkynylene.

The term ‘arylene’ includes divalent aromatic groups, such phenylene or naphthylene. In general, the arylene groups may be monocyclic or polycyclic fused ring aromatic groups. Preferred arylene are C₆-C₁₄arylene.

Other examples of arylene groups are divalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene.

The term ‘heterohydrocarbyl’ includes hydrocarbyl groups in which up to three carbon atoms, preferably up to two carbon atoms, more preferably one carbon atom, are each replaced independently by O, S, Se or N, preferably O, S or N. Heterohydrocarbyl groups thus include heteroalkyl, heteroalkenyl and heteroalkynyl groups, cycloheteroalkyl (including polycycloheteroalkyl), cycloheteroalkenyl and heteroaryl groups and combinations thereof, e.g. heteroalkylcycloalkyl, alkylcycloheteroalkyl, heteroalkylpolycycloalkyl, alkylpolycycloheteroalkyl, heteroalkylaryl, alkylheteroaryl, heteroalkenylaryl, alkenylheteroaryl, cycloheteroalkylaryl, cycloalkylheteroaryl, heterocycloalkenylaryl, cycloalkenylheteroaryl, cycloalkylheteroalkyl, cycloheteroalkylalkyl, polycycloalkylheteroalkyl, polycycloheteroalkylalkyl, arylheteroalkyl, heteroarylalkyl, arylheteroalkenyl, heteroarylalkenyl, arylcycloheteroalkyl, heteroarylcycloalkyl, arylheterocycloalkenyl and heteroarylcycloalkenyl groups. The heterohydrocarbyl groups may be attached to the remainder of the compound by any carbon or hetero (e.g. nitrogen) atom.

The term ‘heterohydrocarbylene’ includes hydrocarbylene groups in which up to three carbon atoms, preferably up to two carbon atoms, more preferably one carbon atom, are each replaced independently by O, S, Se or N, preferably O, S or N. Heterohydrocarbylene groups thus include heteroalkylene, heteroalkenylene and heteroalkynylene groups, cycloheteroalkylene (including polycycloheteroalkylene), cycloheteroalkenylene and heteroarylene groups and combinations thereof, e.g. heteroalkylenecycloalkylene, alkylenecycloheteroalkylene, heteroalkylenepolycycloalkylene, alkylenepolycycloheteroalkylene, heteroalkylenearylene, alkyleneheteroarylene, heteroalkenylenearylene, alkenyleneheteroarylene, cycloalkyleneheteroalkylene, cycloheteroalkylenealkylene, polycycloalkyleneheteroalkylene, polycycloheteroalkylenealkylene, aryleneheteroalkylene, heteroarylenealkylene, aryleneheteroalkenylene, heteroarylenealkenylene groups. The heterohydrocarbylene groups may be attached to the remainder of the compound by any carbon or hetero (e.g. nitrogen) atom.

Where reference is made to a carbon atom of a hydrocarbyl or other group being replaced by an O, S, Se or N atom, what is intended is that:

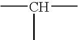

is replaced by

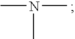

—CH═0 is replaced by —N═; or
—CH₂— is replaced by —O—, —S— or —Se—.

The term ‘heteroalkyl’ includes alkyl groups in which up to three carbon atoms, preferably up to two carbon atoms, more preferably one carbon atom, are each replaced independently by O, S, Se or N, preferably O, S or N.

The term ‘heteroalkenyl’ includes alkenyl groups in which up to three carbon atoms, preferably up to two carbon atoms, more preferably one carbon atom, are each replaced independently by O, S, Se or N, preferably O, S or N.

The term ‘heteroalkynyl’ includes alkynyl groups in which up to three carbon atoms, preferably up to two carbon atoms, more preferably one carbon atom, are each replaced independently by O, S, Se or N, preferably O, S or N.

The term ‘heteroaryl’ includes aryl groups in which up to three carbon atoms, preferably up to two carbon atoms, more preferably one carbon atom, are each replaced independently by O, S, Se or N, preferably O, S or N. Preferred heteroaryl are C₅₋₁₄heteroaryl. Examples of heteroaryl are pyridyl, pyrrolyl, thienyl or furyl.

Other examples of heteroaryl groups are monovalent derivatives of acridine, carbazole, β-carboline, chromene, cinnoline, furan, imidazole, indazole, indole, indolizine, isobenzofuran, isochromene, isoindole, isoquinoline, isothiazole, isoxazole, naphthyridine, perimidine, phenanthridine, phenanthroline, phenazine, phthalazine, purine, pyran, pyrazine, pyrazole, pyridazine, pyridine, pyrimidine, pyrrole, pyrrolizine, quinazoline, quinoline, quinolizine, quinoxaline, thiophene and xanthene. Preferred heteroaryl groups are five- and six-membered monovalent derivatives, such as the monovalent derivatives of furan, imidazole, isothiazole, isoxazole, pyran, pyrazine, pyrazole, pyridazine, pyridine, pyrimidine, pyrrole, pyrrolizine and thiophene. The five-membered monovalent derivatives are particularly preferred, i.e. the monovalent derivatives of furan, imidazole, isothiazole, isoxazole, pyrazole, pyrrole and thiophene.

The term ‘heteroalkylene’ includes alkylene groups in which up to three carbon atoms, preferably up to two carbon atoms, more preferably one carbon atom, are each replaced independently by O, S, Se or N, preferably O, S or N.

The term ‘heteroalkenylene’ includes alkenylene groups in which up to three carbon atoms, preferably up to two carbon atoms, more preferably one carbon atom, are each replaced independently by O, S, Se or N, preferably O, S or N.

The term ‘heteroalkynylene’ include alkynylene groups in which up to three carbon atoms, preferably up to two carbon atoms, more preferably one carbon atom, are each replaced independently by O, S, Se or N, preferably O, S or N.

The term ‘heteroarylene’ includes arylene groups in which up to three carbon atoms, preferably up to two carbon atoms, more preferably one carbon atom, are each replaced independently by O, S, Se or N, preferably O, S or N. Preferred heteroarylene are C₅₋₁₄heteroarylene. Examples of heteroarylene are pyridylene, pyrrolylene, thienylene or furylene.

Other examples of heteroarylene groups are divalent derivatives (where the valency is adapted to accommodate the q instances of the linker L) of acridine, carbazole, β-carboline, chromene, cinnoline, furan, imidazole, indazole, indole, indolizine, isobenzofuran, isochromene, isoindole, isoquinoline, isothiazole, isoxazole, naphthyridine, perimidine, phenanthridine, phenanthroline, phenazine, phthalazine, purine, pyran, pyrazine, pyrazole, pyridazine, pyridine, pyrimidine, pyrrole, pyrrolizine, quinazoline, quinoline, quinolizine, quinoxaline, thiophene and xanthene. Preferred heteroarylene groups are five- and six-membered divalent derivatives, such as the divalent derivatives of furan, imidazole, isothiazole, isoxazole, pyran, pyrazine, pyrazole, pyridazine, pyridine, pyrimidine, pyrrole, pyrrolizine and thiophene. The five-membered divalent derivatives are particularly preferred, i.e. the divalent derivatives of furan, imidazole, isothiazole, isoxazole, pyrazole, pyrrole and thiophene.

### Substitution

The above indicated groups may be substituted with one or more substituents selected from the group consisting of halogen, trihalomethyl, —NO₂, —CN, —N⁺(R¹)₂O⁻, —CO₂H, —CO₂R¹, —SO₃H, —SOR¹, —SO₂R¹, —SO₃R¹, —OC(═O)OR¹, —C(═O)H, —C(═O)R¹, —OC(═O)R¹, —NR¹₂, —C(═O)NH₂, —C(═O)NR¹₂, —N(R¹)C(═O)OR¹, —N(R¹)C(═O)NR¹₂, —OC(═O)NR¹₂, —N(R¹)C(═O)R¹, —C(═S)NR¹₂, —NR¹C(═S)R¹, —SO₂NR¹₂, —NR¹SO₂R¹, —N(R¹)C(═S)NR¹₂, —N(R¹)SO₂NR¹₂, —R¹ or -Z²R¹.

Z² is O, S, Se or NR¹.

R¹ is independently H, C₁₋₈hydrocarbyl, C₁₋₈hydrocarbyl substituted with one or more S_{ub}¹, C₁₋₈heterohydrocarbyl or C₁₋₈heterohydrocarbyl substituted with one or more S_{ub}¹.

S_{ub}¹ is independently halogen, trihalomethyl, —NO₂, —CN, —N⁺(C₁₋₆alkyl)₂O⁻, —CO₂H, —CO₂C₁₋₆alkyl, —SO₃H, —SOC₁₋₆alkyl, —SO₂C₁₋₆-alkyl, —SO₃C₁₋₆alkyl, —OC(═O)OC₁₋₆alkyl, —C(═O)H, —C(═O)C₁₋₆alkyl, —OC(═O)C₁₋₆alkyl, —N(C₁₋₆alkyl)₂, —C(═O)NH₂, —C(═O)N(C₁₋₆alkyl)₂, —N(C₁₋₆alkyl)C(═O)O(C₁₋₆alkyl), —N(C₁₋₆alkyl)C(═O)N(C₁₋₆alkyl)₂, —OC(═O)N(C₁₋₆alkyl)₂, —N(C₁₋₆-alkyl)C(═O)C₁₋₆-alkyl, —C(═S)N(C₁₋₆alkyl)₂, —N(C₁₋₆alkyl)C(═S)C₁₋₆alkyl, —SO₂N(C₁₋₆alkyl)₂, —N(C₁₋₆alkyl)SO₂C₁₋₆alkyl, —N(C₁₋₆-alkyl)C(═S)N(C₁₋₆alkyl)₂, —N(C₁₋₆alkyl)SO₂N(C₁₋₆alkyl)₂, C₁₋₆alkyl or -Z²C₁₋₆alkyl.

Where reference is made to a substituted group, the substituents are preferably from 1 to 5 in number, most preferably 1.

### General

The term “comprising” encompasses “including” as well as “consisting” e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

### EXAMPLES

### Example 1 (1,3,5,7-Tetrakis(4-(4-hydroxybutyn-1-yl)phenyl)adamantane)

A suspension of 1,3,5,7-tetrakis(4-iodophenyl)adamantane (7.55 g, 8 mmol) in DMF (100 mL) was degassed by altering between vacuum and argon for three times. Butyn-4-ol (3.03 mL, 40 mmol), triethylamine (6.68 mL, 48 mmol), tetrakis(triphenylphosphine)palladium (462 mg, 0.4 mmol) and copper (I) iodide (154 mg, 0.8 mmol) were added consecutively. The flask was flushed with argon; the mixture was stirred for 12 h, and evaporated. The residue was dissolved in THF (100 mL) and applied onto a column containing silica gel (5 cm thick layer). The product was eluted with THF (500 mL), and the solvent was evaporated. The residue was suspended in methanol (100 mL), filtered off and washed with methanol (3×50 mL), hot toluene (3×50 mL) and dichloromethane (3×50 mL), and dried in vacuo. Yellowish solid. Yield 3.69 g (65%).

### Example 2

### (1,3,5-Tris(4-(4-dimethoxytrityloxybutyn-1-yl)phenyl)-7-(4-(4-hydroxybytyn-1-yl)phenyl)adamantane

Tetraol (1.5 g; 2.10 mmol) was co-evaporated with anhydrous pyridine (2×50 mL) dissolved in anhydrous pyridine (100 mL) and cooled to 0° C. follow 4,4.-dimethoxytrityl chloride (2.14 g, 6.31 mmol) was added by three portions. The reaction mixture was left for 12 h. TLC showed disappearance of starting material. The reaction mixture was quenched with methanol (3 mL) evaporated about two third diluted with ethyl acetate (300 mL) and successively washed with 5% NaHCO3 (2×100 mL), water (100 mL) and brine (100 mL). Organic layer was dried over Na2SO4 evaporated, and residue was separated on silica gel (column h=20 cm; d=5 cm). Rf 0.39 (EtOAc:PhMe 1:4+1% Et3N). Yield 1.2 g (35%).

### Example 3

### (1,3,5-Tris(4-(4-dimethoxytrityloxybutyn-1-yl)phenyl)-7-(4-(4-[2-cyanoethoxy-N,Ndiisopropylaminophosphinyloxy]butyn-1-yl)phenyl)adamantane)

The alcohol was co-evaporated with anhydrous freshly distilled DCM (2×40 mL) dissolved in anhydrous DCM (60 mL) and evaporated about one third. 2-Cyanoethyl-N,N,N,N.-tetraisopropylphosphorodiamidite (325 μL, 1.02 mmol) and diisopropylammonium tetrazolide (175 mg, 1.02 mmol) were added to the solution of alcohol and reaction mixture was left for overnight. TLC showed complete conversion of starting material. The reaction mixture was diluted with ethyl acetate successively washed with 5% NaHCO3 (2×100 mL) and brine (100 mL). Organic layer was dried over Na2SO4 evaporated, and residue was precipitated into hexane (400 mL). The phosphoroamidite was lyophilized from benzene. Rf 0.63 (EtOAc:PhMe; 1:4+1% Et3N). Yield 1.05 g (85%).

### Example 4

### (1-[4-(4-hydroxy-1-butyn-1-yl)phenyl]-3,5,7-tris(4-iodophenyl)adamantane)

A solution of 1,3,5,7-tetrakis(4-iodophenyl)adamantane (770 mg, 0.816 mmol) in DMF (15 ml) was degassed tree times by altering between vacuum and argon. 3-Butynol (68 μL, 0.897 mmol), triethylamine (0.23 mL, 1.632 mmol), tetrakis(triphenylphosphine)palladium(0) (94 mg, 0.081 mmol) and copper(I) iodide (31 mg, 0.163 mmol) were added consecutively. The mixture was stirred for six hours, diluted with EtOAc (50 mL) and poured into EtOAc (100 mL) and water (100 mL). The organic layer was washed with water (3×50 mL), 0.5 M aqueous disodium EDTA (2×50 mL), water (2×50 mL), and brine (50 mL), dried over Na2SO4 and evaporated. The residue was chromatographed on silica gel (5% EtOAc in PhMe) to provide the title compound as a yellowish foam, Rf 0.31 (10% EtOAc in PhMe). Yield 283 mg (39%). 1H NMR (δ, DMSO-d6): 2.00, 2.01 (br.s., 12H, adamantane CH2), 2.54 (t, 2H, CH2C≡C, 3J=6.9), 3.57 (app. q, 2H, CH2OH), 4.85 (t, 1H, OH, 3J=5.6), 7.35 (d, 2H, CHAr, 3J=8.5), 7.37 (d, 6H, CHAr, 3J=8.5), 7.51 (d, 2H, CHAr, 3J=8.5), 7.67 (d, 6H, CHAr, 3J=8.5).

### Example 5

### (N-(4-methoxytrityl)propargylamine)

Propargylamine (1.03 mL, 15 mmol) and triethylamine (4.17 mL, 30 mmol) were dissolved in pyridine (30 mL) and 4-methoxytrityl chloride (4.632 g, 15 mmol) was added portionwise. The mixture was stirred for 2 h, diluted with EtOAc (50 mL) and poured into EtOAc (150 mL) and water (200 mL). The organic layer was washed with sat. aqueous NaHCO3 (3×100 mL), water (2×100), and brine (50 mL), dried over Na2SO4 and evaporated. The residue was chromatographed on silica gel (1.5% Et3N in PhMe). The title compound was obtained as yellowish oil. Rf 0.82 (1.5% Et3N in PhMe). Yield 4.53 g (92%). 1H NMR (6, DMSO-d6): 2.72 (dd, 2H, CH2, 3J=7.8, 4J=2.3), 3.03 (t, 1H, ≡CH, 4J=2.3), 3.19 (t, 1H, NH, 3J=7.8), 3.72 (s, 3H, OMe), 6.86 (d, 2H, CHAr, 3J=8.9), 7.18 (t, 2H, p-CHAr, 3J=7.3), 7.26-7.32 (m, 6H, CHAr), 7.39 (d, 4H, CHAr, 3J=7.3).

### Example 6

### (1-[4-(4-hydroxy-1-butyn-1-yl)phenyl]-3,5,7-tris[4-(3-N-(4-methoxytrityl)amino-1-propynyl)phenyl]adamantane)

A solution of 1-[4-(4-hydroxy-1-butyn-1-yl)phenyl]-3,5,7-tris(4-iodophenyl)adamantane (258 mg, 0.291 mmol) and N-(4-methoxytrityl)propargylamine (314 mg, 0.961 mmol) in DMF (4 mL) was degassed tree times by altering between vacuum and argon. Triethylamine (0.24 mL, 1.746 mmol), tetrakis(triphenylphosphine)palladium(0) (34 mg, 0.029 mmol) and copper(I) iodide (11 mg, 0.058 mmol) were added consecutively. The mixture was stirred for 12 h, diluted with EtOAc (50 mL) and poured into EtOAc (100 mL) and water (100 mL). The organic layer was washed with water (3×50 mL), 0.5 M aqueous disodium EDTA (2×50 mL), water (2×50 mL), and brine (50 mL), dried over Na2SO4 and evaporated. The residue was chromatographed on silica gel (5% EtOAc an 1.5% Et3N in PhMe) to provide the title compound as yellowish foam, Rf 0.39 (10% EtOAc and 1.5% Et3N in PhMe). Yield 405 mg (86%). 1H NMR (δ, DMSO-d6): 2.06 (br. s., 12H, adamantane CH2), 2.55 (t, 2H, CH2C≡C, 3J=7.0), 3.03 (d, 6H, CH2NH, 3J=6.9), 3.25 (t, 3H, NH, 3J=6.9), 3.57 (app. q, 2H, CH2OH), 3.72 (s, 9H, MeO), 4.83 (t, 1H, OH, 3J=5.5), 6.86 (d, 6H, CHAr, 3J=8.9), 7.19 (t, 6H, CHAr, 3J=7.4), 7.26-7.36 (m, 26H, CHAr), 7.43 (d, 12H, CHAr, 3J=7.6), 7.51-7.55 (m, 8H, CHAr).

### Example 7

### (1-[4-(4-(2-cyanoethoxy)diisopropylaminophosphinyloxy-1-butyn-1-yl)phenyl]-3,5,7-tris[-4-(3-N-(4-methoxytrityl)amino-1-propynyl)phenyl]adamantane)

Diisopropylammonium tetrazolide (69 mg, 0.404 mmol was added to a stirred solution of 1-[4-(4-hydroxy-1-butyn-1-yl)phenyl]-3,5,7-tris[4-(3-N-(4-methoxytrityl)amino-1-propynyl)phenyl]adamantane (400 mg, 0.269 mmol) in dry DCM (20 mL) under argon. Cyanoethoxybis-(diisopropylamino)-phosphine (123 mg, 0.404 mmol) was added subsequently. The mixture was stirred for 12 h, diluted with DCM (50 mL) and poured into sat. aqueous NaHCO3 (100 mL) and DCM (100 mL). The organic layer was washed with sat. aqueous NaHCO3 (2×50 mL) and water (2×50), dried over Na2SO4 and evaporated. The residue was very quickly chromatographed on silica gel (1.5% Et3N in PhMe). The title compound was obtained as colourless oil. Yield 312 mg (69%). 1H NMR (6, DMSO-d6): 1.10-1.17 (m, 14H, (CH3)2CH), 2.04 (br. s., 12H, adamantane CH2), 2.65-2.79 (m, 4H, CH2CN and CH2C≡C), 3.02 (d, 6H, CH2NH, J=7.3), 3.27 (d, 3H, NH, 3J=7.3), 3.55-3.64 (m, 2H, CH2OH), 3.72 (s, 9H, OMe), 3.74-3.82 (m, 2H, OCH2CH2CN), 6.86 (d, 6H, CHAr, 3J=8.9), 7.19 (t, 6H, CHAr, 3J=7.3), 7.26-7.36 (m, 26H, CHAr), 7.43 (d, 12H, CHAr, 3J=7.6), 7.52 (app. d, 8H, CHAr). 31P NMR (δ, DMSO-d6):147.2.

1. A dendrimer having a multimeric structure, obtainable by the reaction of a core molecule with branching synthons and terminating synthons, wherein the dendrimer has the formula (1):
X—[(Y)ₙ-(Z)ₘ]ₚ (1)
wherein X is a core molecule having an adamantoid structure;
Y is a branching synthon;
Z is a terminating synthon;
n and m are independently integers having a value in the range from 1 to 50; and
p is an integer in the range from 1 to 10.

2. A dendrimer according to claim 1, wherein the branching synthon Y is obtained by reacting a synthon of formula (2) with the core molecule (X) or a growing branch of the dendrimer:
(P¹)F¹-L¹-J[-L²-F²(P²)]_{q} (2)
wherein F¹P¹ and F²P² are each independently an optionally protected functional group;
L¹ and L² are linker groups;
J is a junction group; and
q is independently an integer in the range from 2 to 10.

3. A dendrimer according to claim 2, wherein the terminating synthon Z is obtained by reacting a synthon of formula (2), wherein F¹P¹, F¹P², L¹, L² and J are as defined in claim 2 and q is independently an integer in the range from 1 to 10, with X—(Y)ₙ or a growing branch of the dendrimer.

4. A dendrimer according to claim 2, wherein at least one of L¹ and L² contains an alkenylene, heteroalkenylene, alkynylene, heteroalkynylene, arylene or heteroarylene group

5. A dendrimer according to claim 1, wherein X is adamantane.

6. A dendrimer according to claim 1, having a branch span of greater than 15 Å.

7. A dendrimer according to claim 1, wherein the branching synthon(s) Y contain an aryl ethynyl group.

8. A dendrimer according to claim 1, wherein not all of the branches [(Y)ₙ-(Z)ₘ] are of the same length, such that the surface of the dendrimer is concave or convex.

9. A dendrimer according to claim 2, wherein at least one of the linker groups L¹ and L² is cleavable.

10. A dendrimer according to claim 9, wherein at least one of the linker groups L¹ and L² is alkoxy.

11. A dendrimer according to claim 1, wherein each unit in at least one branch is labelled.

12. A dendrimer according to claim 2, wherein P¹ and P² are selected from the group consisting of a dimethoxytrityl group, a fluorenylmethoxy-carbonyl group or a levulinyl group.

13. A dendrimer according to claim 2, wherein J is a carbon atom.

14. A dendrimer according to claim 1, having outer ends that terminate with a polynucleotide sequence.

15. A dendrimer according to claim 14, wherein the nucleotide sequence is poly-A, poly-T, poly-G or poly-C.

16. A dendrimer according to claim 1, having outer ends that carry a plurality of different functional groups and/or differently blocked functional groups.

17. A dendrimer according to claim 1, which is 1-[4-(4-hydroxy-1-butyn-1-yl)phenyl]-3,5,7-tris[4-(3-N-(4-methoxytrityl)amino-1-propynyl)phenyl]-adamantine.

18. A dendrimer according to claim 1, which is 1,3,5,7-Tetrakis(4-(4-hydroxybytyn-1-yl)phenyl)adamantane.

19. A dendrimer according to claim 1, which is 1,3,5-Tris(4-(4-dimethoxytrityloxybytyn-1-yl)phenyl)-7-(4-(4-hydroxybytyn-1-yl)phenyl)adamantane.

20. A dendrimer according to claim 1, immobilised on to a support.

21. A library of ligands, the ligands being bound to a dendrimer as defined in claim 1.

22. A method of hybridisation wherein a nucleic acid sample is contacted, under hybridising conditions, with a dendrimer as defined in claim 1, having outer ends which terminate with a polynucleotide sequence and which is immobilised on a solid support to bind the nucleic acid sample of interest to the polynucleotide ends of the dendrimer.
